(19) United States
(12) Patent Application Publication
(10) Pub. No.: US 2017/0196647 A1
(43) Pub. Date: Jul. 13, 2017

(54) Method and Apparatus Pertaining to a Retroactive Application of a Flexible Sheet of Antimicrobial Copper

(71) Applicant: Medline Industries, Inc., Northfield, IL (US)

(72) Inventor: Richard S. Cotey, Green Oaks, IL (US)

(21) Appl. No.: 15/471,320

(22) Filed: Mar. 28, 2017

Related U.S. Application Data
(63) Continuation of application No. 13/628,508, filed on Sep. 27, 2012.

(51) Int. Cl. A61B 46/10; B32B 38/10; A01N 25/34; A61G 1/01; A61G 1/04; A01N 59/20; B32B 37/12; A61M 5/14

(52) U.S. Cl. CPC A61B 46/10; B32B 37/12; B32B 38/10; A61M 5/1417; A61G 1/01; A61G 1/04; A01N 59/20; A01N 25/34; B32B 2307/748; B32B 2311/12; B32B 2535/00

(57) ABSTRACT

An apparatus comprises a flexible sheet comprised of antimicrobial copper that is configured to retroactively wrap conformably about at least one predetermined touch surface. The apparatus can further include a securement mechanism that is configured to secure that flexible sheet to the predetermined touch surface.

### CROSS REFERENCE TO RELATED APPLICATIONS

This application is a continuation of and claims benefit of U.S. patent application Ser. No. 13/628,508 filed Sep. 27, 2012, the content of which is fully incorporated herein by this reference.

### TECHNICAL FIELD

This invention relates generally to antimicrobial copper.

### BACKGROUND

The antimicrobial properties of copper and copper alloys are well known in the art. These materials have a documented ability to kill microbes that contact such materials. This action often occurs within two hours of sustained contact. The United States Environmental Protection Agency has registered hundreds of different copper alloys as “antimicrobial materials” in recognition of this property. (As used herein, the expression “antimicrobial copper” will be understood to include both pure copper as well as any copper-based alloy that exhibits antimicrobial properties.)

It is also well known that microbes can survive on non-antimicrobial surfaces (such as plastic and many metals) for considerable periods of time (often exceeding thirty days). As a result, touch surfaces can accumulate a variety of harmful microbes (including bacteria, viruses, and so forth). When these touch surfaces are proximal to particularly vulnerable persons (such as patients in a hospital room or residents in a nursing home) a considerable risk exists that such microbes will infect such persons and worsen their circumstances.

In recognition of the foregoing problem and the antimicrobial properties of antimicrobial copper, some touch surfaces for sensitive application settings (such as hospital rooms, nursing homes, and so forth) are now formed using antimicrobial copper. For example, stretchers having the touch surfaces of the rails formed of antimicrobial copper are now available. Unfortunately, notwithstanding the benefits of antimicrobial copper, replacing existing equipment to substitute equipment having antimicrobial copper surfaces can represent an insurmountable economic burden for many facilities and institutions.

### BRIEF DESCRIPTION OF THE DRAWINGS

The above needs are at least partially met through provision of the method and apparatus pertaining to a retroactive application of a flexible sheet of antimicrobial copper described in the following detailed description, particularly when studied in conjunction with the drawings, wherein:

FIG. 1 comprises a perspective view as configured in accordance with various embodiments of the invention;

FIG. 2 comprises a perspective view as configured in accordance with various embodiments of the invention;

FIG. 3 comprises a plan view as configured in accordance with various embodiments of the invention;

FIG. 4 comprises a perspective view as configured in accordance with various embodiments of the invention;

FIG. 5 comprises a perspective view as configured in accordance with various embodiments of the invention;

FIG. 6 comprises a perspective view as configured in accordance with various embodiments of the invention;

FIG. 7 comprises a perspective view as configured in accordance with various embodiments of the invention;

FIG. 8 comprises a perspective view as configured in accordance with various embodiments of the invention;

FIG. 9 comprises a perspective view as configured in accordance with various embodiments of the invention;

FIG. 10 comprises a perspective view as configured in accordance with various embodiments of the invention;

FIG. 11 comprises a perspective view as configured in accordance with various embodiments of the invention;

FIG. 12 comprises a perspective view as configured in accordance with various embodiments of the invention;

FIG. 13 comprises a perspective view as configured in accordance with various embodiments of the invention.

FIG. 14 comprises a perspective view as configured in accordance with various embodiments of the invention;

FIG. 15 comprises a perspective view as configured in accordance with various embodiments of the invention; and

FIG. 16 comprises a perspective view as configured in accordance with various embodiments of the invention.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these various embodiments, an apparatus can comprise a flexible sheet comprised of antimicrobial copper that is configured to retroactively wrap conformably about at least one predetermined touch surface. The apparatus can further include a securement mechanism that is configured to secure that flexible sheet to the predetermined touch surface.

By one approach, the flexible sheet can be size and configured to wrap substantially completely about the predetermined touch surface if desired. The apparatus can further include, if desired, a cushion disposed on the backside of at least a portion of the flexible sheet.

The securement mechanism can comprise, by one approach, one or more mechanical fasteners. By another approach, in lieu of the foregoing or in combination therewith, the securement mechanism can comprise an adhesive disposed on a backside of the flexible sheet (and/or on the backside of the aforementioned cushion). In such a case the apparatus can further comprise a peel-away sheet disposed over the adhesive. So configured, the adhesive can be selectively exposed upon peeling away that peel-away sheet to thereby ready the flexible sheet for the aforementioned retroactive installation on the predetermined touch surface.

These teachings are highly flexible in practice and will accommodate a wide range of sizes, shapes, and thicknesses for the flexible sheet. Similarly, these teachings will accommodate use with a wide variety of touch surfaces including, for example, touch surfaces having a cylindrical shape (such as intravenous (IV) poles), substantially planar touch surfaces (such as table tops), and so forth.

So configured, the benefits of deploying antimicrobial copper in a given application setting can be realized without requiring replacement of existing equipment. Accordingly, the antimicrobial benefits of such a deployment can be realized at a greatly reduced cost.

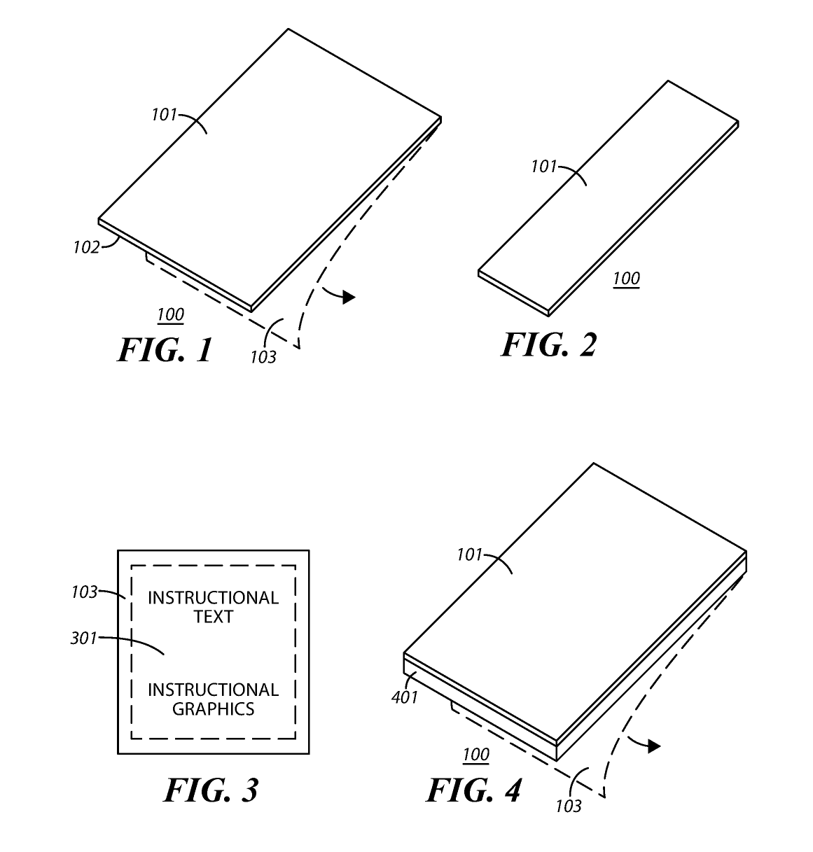

These and other benefits may become clearer upon making a thorough review and study of the following detailed description. Referring now to the drawings, and in particular to FIG. 1, an apparatus 100 that is compatible with many of these teachings will be presented.

This apparatus 100 includes a flexible sheet comprising antimicrobial copper 101. This flexible sheet comprising antimicrobial copper 101 is configured to retroactively wrap conformably about at least one predetermined touch surface. To this end this flexible sheet comprising antimicrobial copper 101 can comprise, for example, a very thin sheet of antimicrobial copper and may even comprise, by way of example, a foil or mesh of such material. Generally speaking, the sheet should be thin enough to readily and easily deform as appropriate to conformally match the surface of the predetermined touch surface.

For many application settings the flexible sheet comprising antimicrobial copper 101 can have an essentially uniform thickness. If desired, however, the thickness can be varied. In such a case, for example, some portion of the flexible sheet comprising antimicrobial copper 101 can be relatively thicker than other portions of the flexible sheet.

The footprint of the flexible sheet comprising antimicrobial copper 101 can vary as desired to meet the needs of a given application setting. FIG. 2, for example, depicts a flexible sheet comprising antimicrobial copper 101 that has both a longer length and a thinner width than the example shown in FIG. 1. These teachings are very flexible in practice and will accommodate essentially any form factor of choice including a variety of circles, ovals, triangles, hexagons, octagons and any of a variety of irregular and/or non-symmetrical shapes as desired.

If desired, the flexible sheet 101 can be secured to one or more other layers of choice to form a corresponding laminate. For example, it may be helpful in some application settings and when working with a particularly thin flexible sheet 101 to secure the flexible sheet 101 to a thin, deformable layer of plastic.

Referring again to FIG. 1, the apparatus 100 also includes a securement mechanism configured to secure the flexible sheet comprising antimicrobial copper 101 to the aforementioned touch surface. By one approach, for example, the securement mechanism can comprise, at least in part, an adhesive disposed on a backside 102 of the flexible sheet comprising antimicrobial copper 101. Such an adhesive can be evenly disposed over that backside 102 if desired or can be limited to some other pattern of partial placement as desired.

The strength of the adhesive can vary as desired as well. For some application settings, for example, it may be useful to employ a relatively weak adhesive to thereby facilitate an intentionally temporary-only deployment of the apparatus 100 (for example, in a temporary sick room at a residence). In other cases it may be important that the adhesive be relatively strong in order to facilitate a permanent installation of the apparatus 100 on the predetermined touch surface.

When the securement mechanism comprises adhesive as described above, the apparatus 100 can further optionally include a peel-away sheet 103 that is disposed over the adhesive. So configured, the peel-away sheet 103 will prevent the adhesive from coming into contact with unintended surfaces until the peel-away sheet 103 is removed. By peeling away that peel-away sheet 103, the adhesive is exposed and the flexible sheet comprising antimicrobial copper 101 is ready to retroactively install on the predetermined touch surface.

That peel-away sheet 103 can be comprised of a material of choice (such as a treated paper or a thin plastic film). Such practices are known in the art. As the present teachings are not particularly sensitive to any particular choices in these regards, further elaboration in these regards are not presented here. Referring momentarily to FIG. 3, however, it will be noted that the peel-away sheet 103 can, if desired, include instructions 301 printed (or otherwise disposed) thereon regarding proper installation of the flexible sheet comprising antimicrobial copper 101 on the predetermined touch surface. These instructions can comprise, for example, instructional text and/or instructional graphics regarding such things as how to prepare the touch surface to receive the apparatus 100, how to remove the peel-away sheet 103, and so forth.

If desired, however, instead of a peel-away sheet 103 an adhesive can be applied at a time of installation on the flexible sheet 101 and/or the predetermined touch surface.

If desired, and referring now to FIG. 4, the apparatus 100 can further optionally include a cushion 401 disposed on the backside 102 of a least a portion of the flexible sheet 101. This cushion 401 can be formed of any of a variety of materials and can have any of a range of thicknesses as appropriate to a particular application setting. The cushion could be, for example, a closed-cell foam that is moisture resistant and would avoid contamination. For many application settings the thickness of the foam could be less than ¼″ of an inch. By one approach the cushion 401 could be permanently attached to the flexible sheet 101. If desired, however, the cushion 401 could remain a physically separate component to facilitate reusing that cushion with other flexible sheets at a later time. When using a cushion 401 the adhesive, when used, can be applied to the backside of the cushion 401 and again a peel-away sheet 103 can be optionally utilized as well if desired.

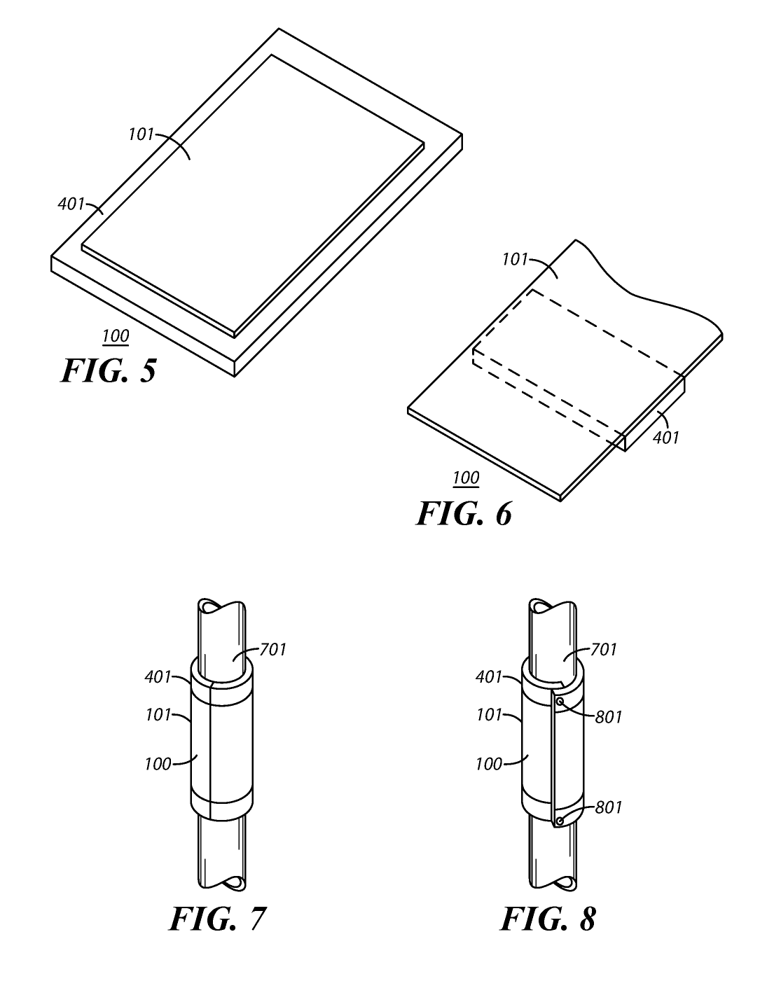

In the illustrative example show in FIG. 4, the cushion 401 is coextensive with the flexible sheet 101. These teachings are highly flexible in these regards, however. As one illustrative example in these regards FIG. 5 depicts an apparatus 100 having a cushion 401 that is both longer and wider than the flexible sheet 101. As another illustrative example in these regards, and referring now to FIG. 6, the apparatus 100 can include a cushion 401 that is only disposed on a portion, but not all, of the backside 102 of the flexible sheet 101. In this particular example the cushion 401 is centrally and laterally disposed with respect to the flexible sheet 101, a configuration that can be useful when applying the apparatus 101, for example, to an edge of a tabletop surface.

As noted earlier, the securement mechanism serves to secure the flexible sheet 101 to a predetermined touch surface. As used herein, the expression “predetermined touch surface” will be understood to refer to a specific form factor or range of touch surface form factors for which the flexible sheet 101 is designed and intended to accommodate. A planar shape as shown in FIG. 1, for example, is suitable for use with flat touch surfaces but will also work simultaneously with, for example, two planar surfaces that are adjoined orthogonally to one another by conformally accommodating both planar surfaces as well as the right angle that joins the two planar surfaces to one another.

As another example in these regards, and referring to FIG. 7, a flexible sheet 101 having a rectangular shape of appropriate dimensions can conformally fit substantially completely about a touch surface comprised, in this example, of a cylinder and, more particularly, an intravenous pole 701. In this particular example, the apparatus 100 includes the aforementioned cushion 401 and is secured to the pole 701 via the aforementioned adhesive. If desired, of course, other securement mechanisms can serve in these same regards. For example, and referring now to FIG. 8, the securement mechanism can comprise one or more mechanical fasteners 801 such as any of a variety of snaps, clips, ties, zippers, hooks-and-loops fasteners, and so forth. Mechanical fasteners 801 that pertain only to the apparatus 100 itself and do not directly interact with the touch surface can be particularly useful when the installation of the apparatus 100 is only intended to be temporary.

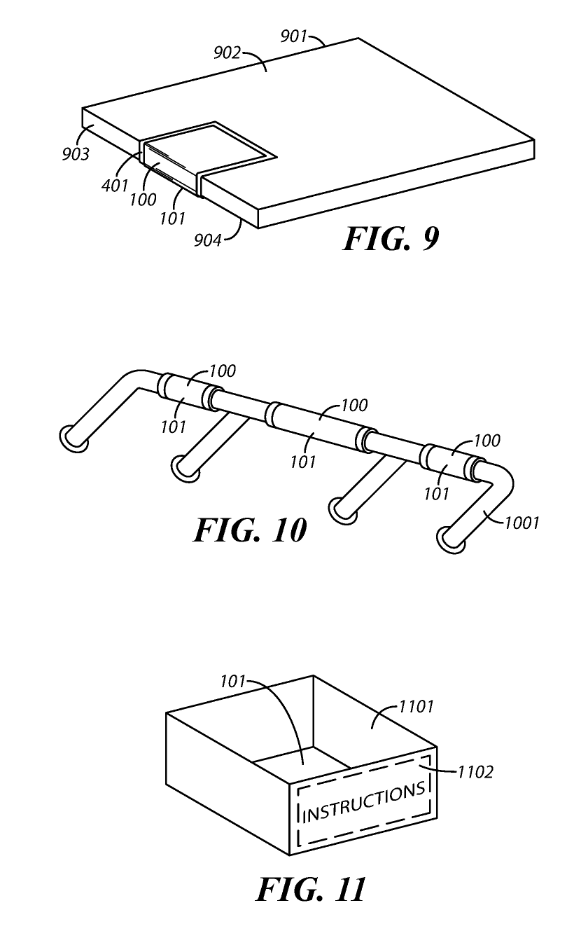

FIG. 9 presents a touch surface comprising a portion of a table 901. In this particular example the apparatus 100 is secured to the top surface 902 of the table 901 and also to a side edge 903 and the underside surface 904 of the table 901 as well. It would of course be possible for the apparatus 100 to similarly cover the entire edge 903 of one or more edges of the table 901 if desired.

As yet another illustrative example as regards the flexible nature of these teachings, FIG. 10 illustrates a stretcher rail 1001 having three separate apparatuses 100 disposed thereon to thereby protect each of a plurality of corresponding touch surfaces.

It will be understood that the foregoing examples are intended to serve only as illustrative examples and are not to be taken as suggesting any particular limitations in these regards. Generally speaking, the apparatus 100 (including specifically the flexible sheet 101) can have essentially any shape and size to thereby accommodate corresponding touch surfaces of interest. Examples of touch surfaces that can be retroactively treated as described herein include, but are not limited to, IV poles, hand rails, table and chair surfaces, floors, stretchers, geriatric chairs, hospital bed rails, over-bed table tops and edges, resident room accessories including push medical carts, cubicle curtain pull wands, emergency carts, hampers, and other mobile or stationary carts and tables and table tops in a surgical environment that are prone to exposure to infection control mandates and that may be listed as a United States Food and Drug Administration (FDA) medical device.

Referring to FIG. 11, by one approach a plurality of such apparatuses 100 can be placed (for example, in a stacked configuration) in a dispensing container 1101 that contains that plurality of apparatuses 100 to facilitate distribution, storage, and dispensation of the individual apparatuses 100. For many application purposes it will be useful for all of the apparatuses 100 in a given container 1101 to be alike. If desired, the container 1101 can include instructions 1102 disposed thereon regarding, for example, proper installation of the flexible sheets 101 that comprise the apparatuses 100.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

As one simple example in these regards, such an apparatus 100 can serve as the basis for a rental business that temporarily provides the apparatuses 100 to persons who are looking to temporarily provide a safer environment in their home for a sick family member. Having served their purpose, the rented apparatuses 100 can be removed from their corresponding touch surfaces and returned to the rental facility.

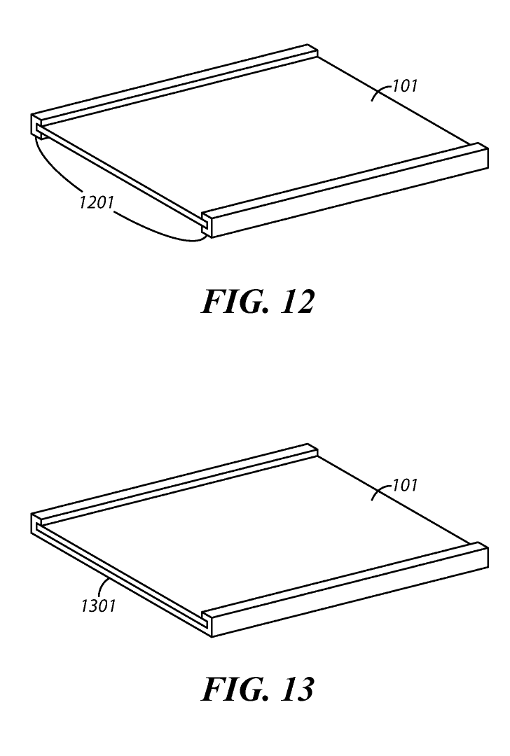

As another example in these regards, and referring to FIGS. 12 and 13, a discrete edge treatment sleeve 1201 can be applied along exposed edges of the sheet 101 or such an edge treatment can be incorporated into a sheet of material 1301 that attaches to the underside of the sheet 101. Plastic or a similar pliable material can be used for this purpose and the edge treatment can be secured using, for example, an appropriate adhesive and/or a friction fit. Such an edge treatment can serve, for example, to cover possibly sharp edges that may be presented by the sheet 101.

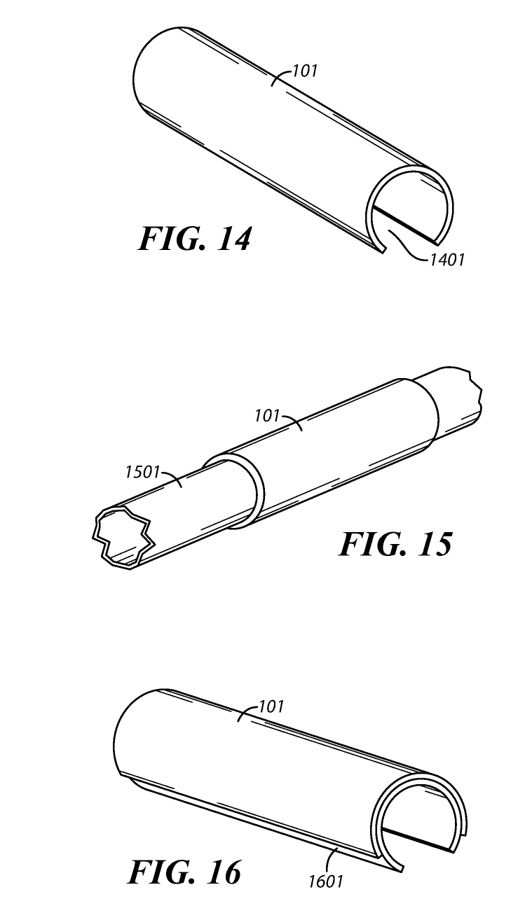

And as yet another example in these regards, the flexible sheet 101 itself can embody the securement mechanism by having spring-like characteristics that, combined with a preferred quiescent form factor, serves to hold the flexible sheet 101 in place on the predetermined touch surface. FIG. 14 provides one illustrative example in these regards where the flexible sheet 101 has a quiescent form factor in the shape of a cylinder having, in this example, a gap 1401 along its longitudinal axis. It would be possible for this gap 1401 to have a variety of dimensions. It would also be possible for longitudinal edges of the flexible sheet 101 to overlap one another in the quiescent state such that there is no gap. Referring to FIG. 15, such a flexible sheet 101 can be disposed around a tubular-shaped predetermined touch surface 1501 at a desired location and retained in the installed position by the spring-like grip imposed by the flexible sheet 101.

As a related approach, and referring now to FIG. 16, the flexible sheet 101 can be affixed (using, for example, a suitable adhesive) to another sheet 1601 of spring-like material (such as a resilient metal or plastic) that has a quiescent form factor suitable to grip a predetermined touch surface of interest.

I claim:

1. A method for retroactively applying an antimicrobial surface to a touch surface on an existing item of equipment, the method comprising:
providing a flexible sheet comprised of antimicrobial copper, the flexible sheet having an adhesive disposed on a backside thereof and a peel-away sheet disposed over the adhesive;
removing the peel-away sheet from the flexible sheet to thereby expose the adhesive;
retroactively conformably wrapping the flexible sheet about a selected touch surface on a cylindrically-shaped part of an existing item of equipment that is deployed for use in an end-user application setting such that the adhesive adheres the flexible sheet to the cylindrically-shaped part with the antimicrobial copper facing outwardly of the selected touch surface.

2. The method of claim 1 wherein the cylindrically-shaped part of an existing item of equipment comprises an intravenous (IV) pole.

3. The method of claim 1 wherein the cylindrically-shaped part of an existing item of equipment comprises a stretcher rail.

4. The method of claim 1 wherein the antimicrobial copper comprises a foil comprised of antimicrobial copper.

5. The method of claim 1 wherein the antimicrobial copper comprises a mesh comprised of antimicrobial copper.

6. The method of claim 1 wherein the peel-away sheet has instructions disposed thereon describing how to properly install the flexible sheet on a selected touch surface of an existing item of equipment.

7. The method of claim 1 wherein the flexible sheet further comprises a cushion layer disposed between the antimicrobial copper and the adhesive.

8. The method of claim 7 wherein the cushion layer comprises closed-cell foam material.

9. The method of claim 1 wherein providing the flexible sheet comprises providing a plurality of the flexible sheets in a container and wherein removing the peel-away sheet from the flexible sheet comprises first removing a particular one of the flexible sheets from the container and then removing the peel-away sheet from that removed flexible sheet.

10. The method of claim 9 wherein providing the plurality of the flexible sheets in the container comprises providing a plurality of the flexible sheets stacked one atop another in the container.
